(19) United States
(12) Patent Application Publication
(10) Pub. No.: US 2015/0093761 A1
(43) Pub. Date: Apr. 2, 2015

(54) Differentiation and amplification method for inducing human neural stem/progenitor cells to differentiate into oligodendrocyte progenitor cells and application thereof

(71) Applicant: Zuo Luan, Beijing (CN)

(72) Inventor: Zuo Luan, Beijing (CN)

(21) Appl. No.: 14/321,748

(22) Filed: Jul. 1, 2014

(30) Foreign Application Priority Data
Sep. 30, 2013 (CN) 201310455895.3
Sep. 30, 2013 (CN) 201310455908.7

(51) Int. Cl. C12N 5/0797

(52) U.S. Cl. CPC C12N 5/0623; C12N 2506/08; C12N 2501/115; C12N 2501/135; C12N 2501/13
USPC 435/7.21; 435/368

(57) ABSTRACT

A Method for inducing human neural stem/progenitor cells to differentiate into oligodendrocyte progenitor cells and application thereof comprises following steps of: pre-treating neural stem cells derived from different resources in pre-treatment medium including bFGF and EGF for culturing; and inducing neural stem cells after pre-treating with inducing medium including PDGF-AA, bFGF and NT3, so as to differentiate into oligodendrocyte progenitor cells (OPCs). Main markers of the OPCs obtained by the method, such as NG2, O4, A2B5 and PDGFR, have a positive rate of 80˜90%. The OPCs obtained thereby is capable of proliferating steadily in the OPCs inducing medium for at least 10 generations and simultaneously maintaining biological characteristics thereof unchanged. The OPCs induced by the present invention can be applied in treating myelin-associated diseases or researching on drug screening.

### CROSS REFERENCE OF RELATED APPLICATION

This application claims priority under 35 U.S.C. 119(a-d) to CN 201310455895.3, filed Sep. 30, 2013, and CN 201310455908.7, filed Sep. 30, 2013.

### BACKGROUND OF THE PRESENT INVENTION

1. Field of Invention

The present invention relates to the field of cell biology and neurobiology, and more particularly to a differentiation and amplification method for inducing human neural stem/progenitor cells to differentiate into oligodendrocyte progenitor cells and application thereof.

2. Description of Related Arts

In various myelin-associated diseases, such as cerebral white matter damage, spinal cord injury and multiple sclerosis, due to dysmyelination disorder and demyelination thereof, neurons is not capable of transferring electrical excitation normally, which leads to function disability such as body exercise, and thus brings great damage to sufferers of these diseases and their families. However, so far, no medicine is capable of curing the myelin-associated diseases effectively.

The root of both the dysmyelination disorder and the demyelination is the loss of myelination of neuronal axon, thus with remyelination of the neuronal axon, currents of neuron is capable of being conducted steadily and rapidly, the diseases mentioned above is capable of being cured in theory. Research shows that oligodendrocyte progenitor cells (OPCs) are critical cells for the myelination of the central nervous system. In the central nervous system, the OPCs can differentiate into oligodendrocyte (OL), wherein the OL surrounds the neuronal axon to form a complete myelin structure, so as to ensure the steady and rapid conduction of neuronal currents. On the one hand, the myelin-associated diseases are due to damages of the already formed myelin structure, and on the other hand, due to damages of the OPCs in the nervous system, the OL loses source and thus the myelin is difficult to regenerate. Animal experiment shows that when OPCs are transplanted into shiver mice with congenital myelination disorder or rat with cerebral white matter damage, the OPCs transplanted thereof are capable of normally surrounding the neuronal axon in bodies thereof to form the myelin to recover neural function of these animals, which indicates that OPCs transplantation may offer new therapeutic approaches in the treatment of myelin damage diseases.

Although OPCs of rodents are easily obtained, acquisition of human OPCs is quite difficult. Although human OPCs can be obtained by various cell isolated techniques, quantity obtained thereof is very limited and far from meeting the demand for clinical application. The study of stem cells provides a new direction for obtaining OPCs. With self-renewal ability and multiple differentiation potentials, stem cell includes embryonic stem cells and various adult stem cells. The embryonic stem cells are capable of differentiating into any kind of body cells, but the adult stem cells are usually only capable of differentiating into cells determined by lineage thereof. Studies from American Hans and et al. have found that the embryonic stem cells can differentiate into OPCs in vitro, and the transplantation of OPCs contributes to neural functional recovery of spinal cord injuries. The method is applied in clinical phase I research for treating spinal cord injuries. However, the totipotency of the embryonic stem cells cuts both ways, which not only is capable of differentiating into various cells, but also has risk of tumorigenesis. Thus, OPCs obtained by the method has a certain risk of tumorigenesis in clinical application. Since differentiation ability of the adult stem cells is limited, which avoids the risk of tumorigenesis to some extent, the adult stem cells is more secure in clinical application. Neural stem/progenitor cells (NS/PCs) are a kind of adult stem cells which are derived from nerve tissue or embryonic stem cells. From the perspective of development, the NS/PCs are precursor cells of OPCs, and thus are theoretically most suitable for inducing into OPCs.

The differentiation condition of the stem cells are closely related to the microenvironment thereof, which is also applied to NS/PCs. NS/PCs are suspended in vitro and are cultured to form spheric structures. These NS/PCs cells are not completely uniform, and different pretreatment leads to different differentiation ability of the NS/PCs cells. E.g., pretreatment by EGF (epidermal growth factor) is capable of increasing differentiation proportion of astrocytes, and pretreatment with bFGF (basic fibroblast growth factor) is capable of increasing differentiation proportion of neurons.

OPCs of human, being cells with unstable state, are easy to further differentiate and mature into OL. However, OL loses migration capability of OPCs, and once OPCs are differentiated into OL in vitro, clinical effects thereof are absolutely lost. Therefore, it is quite important to maintain characteristics of OPCs in vitro. In the literatures available, no reports are found on culturing and proliferating human OPCs in vitro steadily for a long time.

### SUMMARY OF THE PRESENT INVENTION

In view of the disadvantages mentioned above, after a combined induction by a long-time pre-treatment and growth factors, the present invention provides a differentiation and amplification method for inducing human neural stem/progenitor cells to differentiate into oligodendrocyte progenitor cells and application thereof.

In the present invention, it is primarily found that pre-treating with a certain concentration of EGF combined with bFGF is capable of greatly increasing differentiation proportion of OPCs, which further proves that communication between intracellular and extracellular is the key factor for determining the differentiation of stem cells. However, just utilizing EGF combined with bFGF is inadequate for differentiating NS/PCs into OPCs directly, and other growth factors are further required for obtaining a great quantity of OPCs with high purity. In this application, it is found that a combination of three factors PDGF-AA, bFGF and NT3 is sufficient for inducing pre-treated NS/PCs into OPCs, and that none of the three factors is dispensable. Otherwise, the efficiency of differentiation will be greatly decreased.

The present invention provides a differentiation and amplification method for inducing human neural stem/progenitor cells to differentiate into oligodendrocyte progenitor cells, wherein technical solutions thereof comprise following steps of

pre-treating neural stem/progenitor cells (NS/PCs) of human in pre-treatment medium for culturing a preset time,

inducing the NS/PCs after pre-treating with inducing medium, so as to differentiate into high-purity oligodendrocyte progenitor cells (OPCs) which are capable of expressing OPCs makers including O4, A2B5 and NG2,

wherein the inducing medium substantially comprises bFGF, PDGF-AA and NT-3,

wherein the OPCs obtained thereby are capable of proliferating steadily in proliferating medium for at least 10 generations and maintains biological characteristics thereof unchanged,

wherein the proliferating medium substantially comprises bFGF, PDGF-AA, NT3 and sodium lactate.

The differentiation and amplification method for inducing human neural stem/progenitor cells to differentiate into oligodendrocyte progenitor cells, specifically comprises following steps of:

1. dissociating NS/PCs of human into single cells;

2. suspending the single cells in a pre-treatment medium again after washing, wherein cell density thereof is adjusted to 2˜10×10⁵/ml;

3. plating cells suspension into a cell culture flask, and culturing under a condition of 37° C. with 5˜8.5% CO₂ and saturated humidity;

4. renewing a half of the medium every 3˜5 days until the cells are cultured continuously for 7˜12 days;

5. collecting the cells into centrifuge tube, wherein 400 g thereof is processed with centrifugation for 5 minutes for precipitating the cells;

6. removing supernatant, wherein a mass percentage of 0.025% trypsin is applied for digesting the cells, in such a manner that the cells are digested into single cells suspension, after digesting for 10 minutes, a concentration of 1 mg/ml trypsin inhibitor is applied for inhibiting digestion;

7. centrifugating 400 g of suspension for 5 minutes to collect cells;

8. removing supernatant, wherein inducing medium for OPCs are applied to suspend the cells again, and cell density thereof is adjusted to 2˜10×10⁵/ml;

9. plating suspension of cells into a cell culture flask;

10. processing morphological identification on the OPCs and immunofluorescence staining identification on OPCs markers after inducing for 4˜10 days to find a large quantity of cells are adhered on a bottom of the cell culture flask;

11. collecting the OPCs into a centrifuge tube;

12. centrifugating 400 g of collection for 5 minutes to collect cells;

13. removing supernatant, wherein the cells are suspended in proliferating medium again, and cell density thereof is adjusted to 2˜10×10⁵/ml;

14. planting cell suspension into a cell culture flask;

15. renewing the proliferating medium for every 3˜5 days; and

16. after approximately one week when cell confluence of the OPCs reach 80%, processing passage on the OPCs, wherein passage method thereof is the same with the steps 11˜15 mentioned above, wherein the steps are cycled.

Preferably, the pre-treatment medium adopted by the method mentioned above comprises basal medium and additives, wherein the basal medium is commercial Neural Basal Medium or self-prepared DF medium;

wherein the DF medium comprises: DMEM, F12, HEPES and D-glucose, a volume ratio of the DMEM and F12 is (1˜3): 1, a concentration of the HEPES is 10˜20 mmol/L, a concentration of the D-glucose is 1˜2g/ml (mass to volume),

wherein the additives comprise B27, bFGF, EGF, LIF, transferin, progerterone, putrescine, sodium selenite, insulin and heparin, wherein mass concentrations are respectively: 1× of B27, 15-25 ng/ml of EGF, 10-20 ng/ml of bFGF, 7-13 ng/ml of LIF, 50-150 μg/ml of transferin, 10-30 mmol/L of progerterone, 50-150 μmol/L of putrescine, 20-40 mmol/L of sodium selenite, 10-50 μg/ml of insulin and 3-10 μg/ml of heparin.

Preferably, the inducing medium adopted by the method mentioned above comprises basal medium and additives, wherein the basal medium is commercial Neural Basal Medium or self-prepared DF medium;

wherein the DF medium comprises DMEM, F12, HEPES and D-glucose, a volume ratio of the DMEM and F12 is (1˜3): 1, a concentration of the HEPES is 10˜20 mmol/L, a concentration of the D-glucose is 1˜2 g/ml (mass to volume),

wherein the additives comprise: B27, transferin, progerterone, putrescine, sodium selenite, insulin, heparin, sodium lactate, bFGF, PDGF-AA,and NT-3 and and penicillin-streptomycin (optional), wherein mass concentrations are respectively: 1× of B27, 5-20 μg/ml of transferin, 5-20 nmol/L of progerterone, 20-40 μmol/L putrescine, 10-20 nmol/L of sodium selenite, 5-20 μg/ml of insulin, 2-10 μg/ml of herapin, 3-10 mmol/L of sodium lactate, 5-30 ng/ml of bFGF, 5-30 ng/ml of PDGF-AA, 5-30 ng/ml of NT-3 and 100 U/ml of penicillin-streptomycin.

Preferably, the proliferating medium adopted by the method mentioned above comprises basal medium and additives, wherein the basal medium comprises commercial Neural Basal Medium or self-prepared DF medium, and commercial sugar-free Neural Basal Medium, wherein volume ratio of the commercial Neural Basal Medium or the self-prepared DF medium, and the commercial sugar-free Neural Basal Medium is (1˜3): 1;

wherein the DF medium comprises DMEM, F12, HEPES and D-glucose, a volume ratio of the DMEM and F12 is (1˜3): 1, a concentration of the HEPES is 10˜20 mmol/L, a concentration of the D-glucose is 1˜2 g/ml (mass to volume),

wherein the additives comprise B27, sodium lactate, bFGF, PDGF-AA, NT-3, transferin, progerterone, putrescine, sodium selenite, insulin and heparin, wherein mass concentrations are respectively: 1× of B27, 3-10 mmol/L of sodium lactate, 5-25 ng/ml of bFGF, 10-20 ng/ml of PDGF-AA, 5-25 ng/ml of NT-3, 5-50 μg/ml of transferin, 5-20 mmol/L of progerterone, 20-50 μmol/L of putrescine, 10-20mmol/L of sodium selenite, 5-20 μg/ml of insulin and 2-10 μg/ml of heparin.

Preferably, the neural stem/progenitor cells mentioned above are derived from, but not limited to, brain tissue of human, spinal cord tissue, embryonic stem cells or induced pluripotent stem cells (iPS).

The method of the present invention and the OPCs obtained thereby can be applied in research of experiment in vivo or vitro and clinical therapy for treating diseases of nervous system damage.

The OPCs obtained by the method of the present invention can be applied in preparing medicine for treating diseases of nervous system damage.

The diseases of nervous system damage includes myelin-associated diseases including white matter damage, multiple sclerosis, spinal cord injury, and etc.

The present invention has beneficial effects as follows.

1. Thus the method provided by the present invention not only contributes to clinical treatment and drug screening for the myelin-associated diseases, but also to basic research on myelination, which is reflected in the following aspects of:

\textcircled{1} obtaining high-purity OPCs for applying in clinical treatment and research for myelin-associated diseases;

\textcircled{2} providing sufficient amount of OPCs for clinical application by proliferating in vitro;

\textcircled{3} studying regulation mechanism of the developing of OPCs; and

\textcircled{4} providing a drug screening model for the myelin-associated diseases.

2. In the present invention, non-animal-source and chemical pre-treatment medium is combined with OPCs inducing medium to inducing NS/PCs of human into OPCs. The OPCs produced by induction thereof do not contain any animal-resource ingredient, and therefore is capable of avoiding security risks brought by animal-resource ingredients probably existed in cell treatment, which provides a way for treating myelin-associated diseases safely and effectively, and thus has more extensive prospects in clinical application.

3. The OPCs induced by the method of the present invention are capable of proliferating in vitro for at least 10 generations and simultaneously maintaining biological characteristics to proliferate for thousands of times, and thus is capable of providing sufficient quantity of cells for basic or clinical research.

4. The OPCs produced by the method of the present invention can be processed with cryo-preservation or recovery for further culturing, and the biological characteristics maintains unchanged after cryo-preservation recovery, which is further conductive to widely popularizing OPCs transplantation for clinical treatment and research.

These and other objectives, features, and advantages of the present invention will become apparent from the following detailed description, the accompanying drawings, and the appended claims.

### BRIEF DESCRIPTION OF THE DRAWINGS

FIG. 1 is an optical morphology image of OPCs induced by the method of the present invention, which indicates that the OPCs are multipolar or bipolar, and that soma thereof is small.

FIG. 2 is an O4 fluorescent staining image of the OPCs induced by the method of the present invention, and the result is positive.

FIG. 3 is an A2B5 fluorescent staining image of the OPCs induced by the method of the present invention, and the result is positive.

FIG. 4 is an SOX10 fluorescent staining image of the OPCs induced by the method of the present invention, and the result is positive.

FIG. 5 is an NG2 fluorescent staining image of the OPCs induced by the method of the present invention, and the result is positive.

FIG. 6 is a PDGFR fluorescent staining image of the OPCs induced by the method of the present invention, and the result is positive.

FIG. 7 is an optical morphology image of proliferating OPCs of the first generation.

FIG. 8 is an optical morphology image of proliferating OPCs of the third generation.

FIG. 9 is an optical morphology image of proliferating OPCs of the fifth generation.

FIG. 10 is an optical morphology image of proliferating OPCs of the tenth generation.

FIG. 11 is an O4 fluorescent staining image of the proliferating OPCs of the tenth generation, and the result indicates that positive rate of the OPCS reach 80˜90%.

FIG. 12 is a fluorescent staining image for a neuronal marker Tuj-1 of the proliferating OPCs of the tenth generation.

FIG. 13 is a fluorescent staining image for a GFAP of the proliferating OPCs of the tenth generation.

### DETAILED DESCRIPTION OF THE PREFERRED EMBODIMENT

Further description of the technical solution of the present invention is illustrated combining with the following preferred embodiments and the accompanying drawings, which is not intended to be limiting.

### EXAMPLE 1

In this example, a first cell line of NSCs formed in our laboratory were utilized for inducing into OPCs. The NSCs were derived from hippocampus of abandoned embryos. The NSCs were passaged to a tenth generation.

I. Induction

1. NSCs were digested into single cells, washed and suspended in NS/PCs medium, suspension of the cells was planted into a T25 cell culture flask according to a cell density of 2×10⁶/T25, and cultured under 37° C. with 8.5% CO₂ and saturated humidity.

2. Half of the medium was renewed on the fourth day of cell culture.

3. Half of the medium was renewed on the eighth day of the cell culture.

4. On the twelfth day of the cell culture, the cells were collected into a centrifuge tube, and 400 g thereof was centrifuged for 5 minutes.

5. 0.025% trypsin was applied for digesting the cells, in such a manner that the cells were digested into single cells suspension, trypsin inhibitor was applied for inhibiting digestion, and the cells were blowed and beated into single-cell suspension.

6. Centrifuged to collect cells, and centrifugation condition was 400 g for 5 minutes.

7. Inducing medium for OPCs was applied to suspend the cells again, and was planted into a new cell culture flask according to a cell density of 2˜10×10⁵/ml.

8. The suspension of cells was induced for 5 days, and a large quantity of adhered cells was found on a bottom of the cell culture flask.

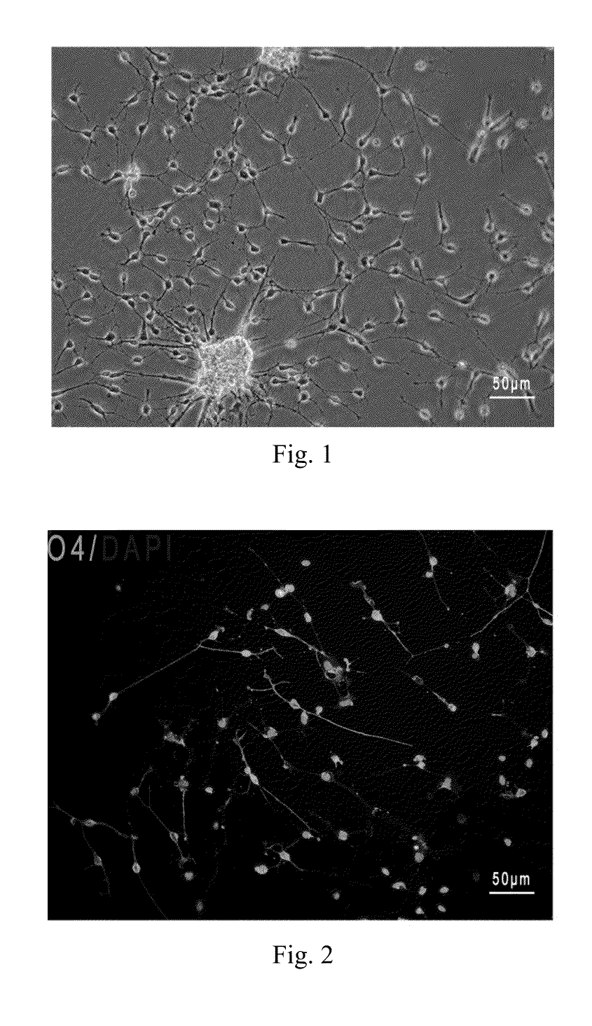

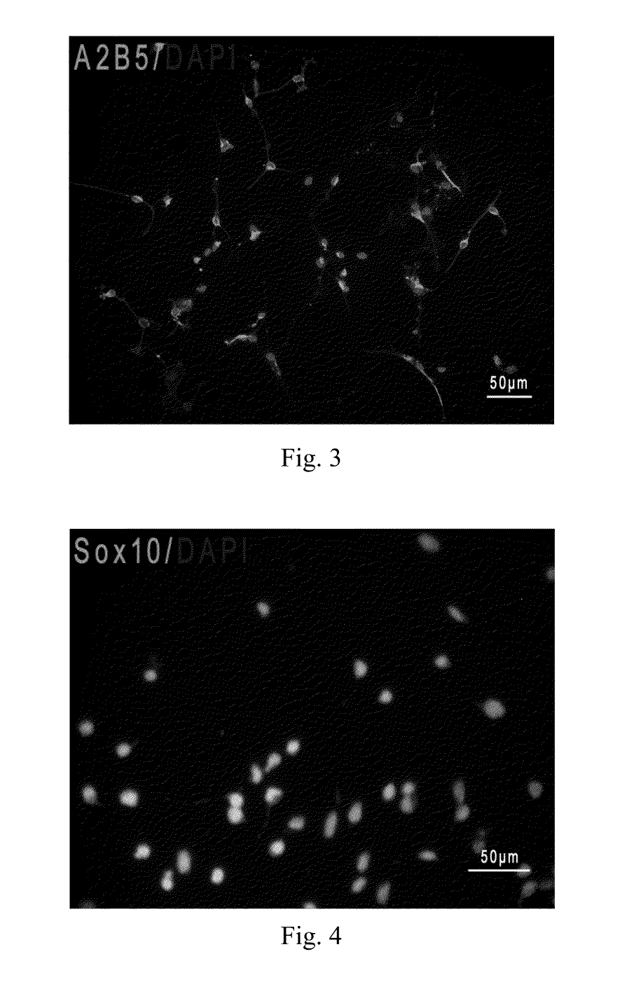

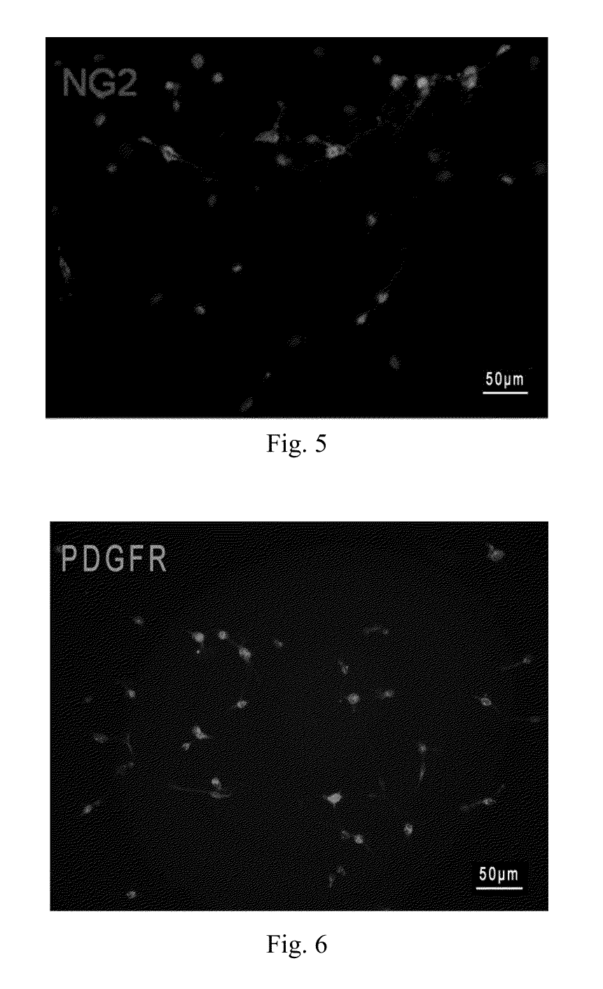

9. OPCs identification, comprising:
1) morphological identification, wherein the results were shown in FIG. 1 of the drawings; and
2) immunofluorescence staining identification for OPCs markers including O4, A2B5 SOX10, NG2 and PDGFR, wherein the results were shown in FIGS. 2˜6 of the drawings.

The method of the OPCs identification was as same as a conventional method of cell identification. The results show that the OPCs were bipolar or multipolar, and expression of the OPCs for markers thereof such as O4, A2B5, SOX10, NG2 and PDGFR was high, and positive rate thereof is 80˜90%.

II. Amplification

1. The OPCs obtained by the method mentioned above were processed with passage operation when confluence thereof was approximately 80%.

2. The OPCs were beated upon slightly by a pasteur pipet, in such a manner that the OPCs were exfoliated.

3. The OPCs were collected into a centrifuge tube, and 400 g thereof was centrifuged for 5 minutes for collecting.

4. Supernatant was removed, wherein the cells were suspended in proliferating medium again, and cell density thereof was adjusted to 2˜10×10⁵/ml. Cell suspension was planted into a cell culture flask.

5. The proliferating medium was renewed for every 3˜5 days.

6. After approximately one week when cell confluence of the OPCs reached 80% , the OPCs were processed with passage, wherein passage method thereof was the same with the steps 1˜5 mentioned above, wherein the steps were cycled.

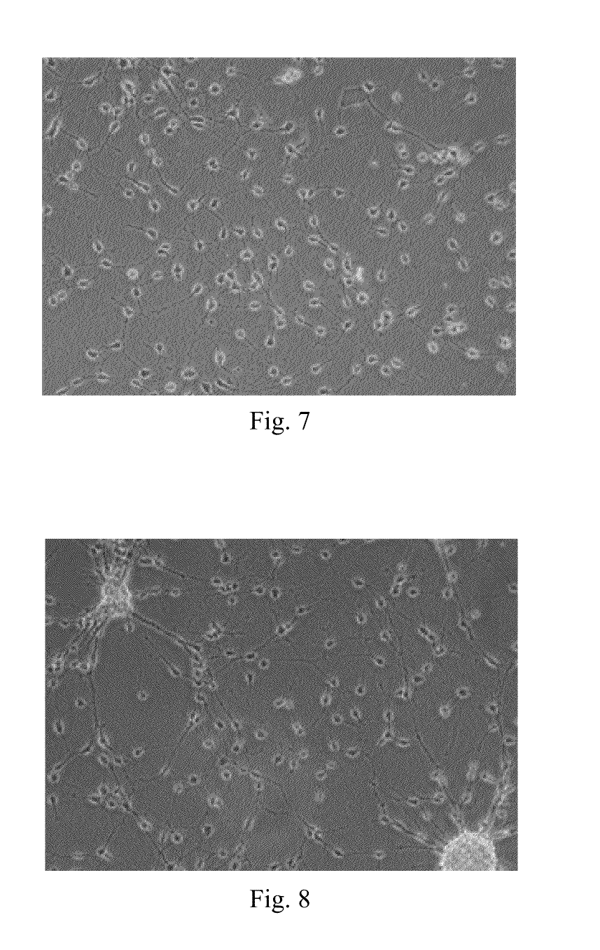

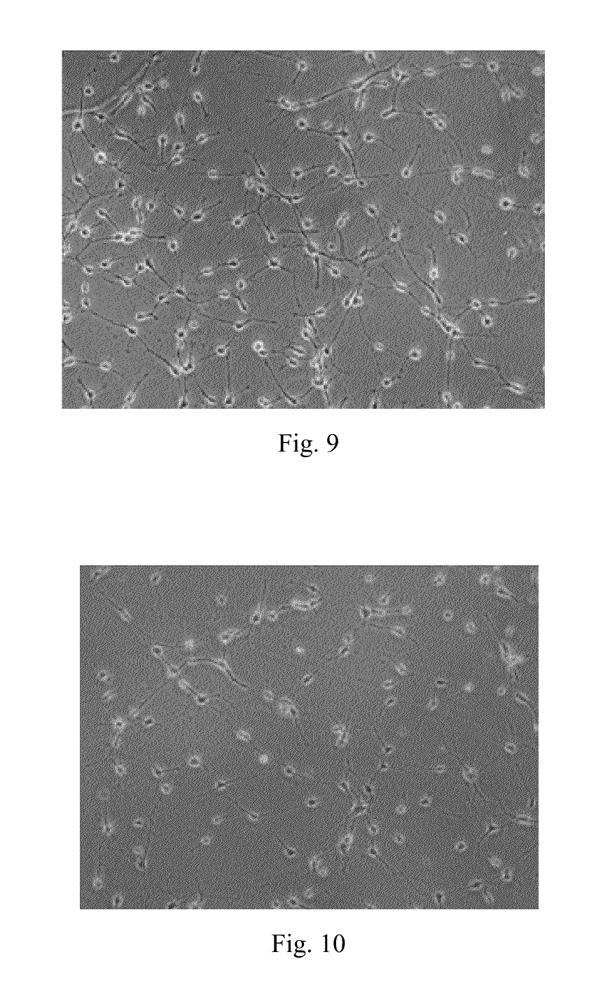

7. The OPCs of the first, third, fifth and tenth generations obtained by the amplification were respectively processed with optical identification. As shown in FIGS. 7˜10 of the drawings, the results showed that morphology of the OPCs remained unchanged.

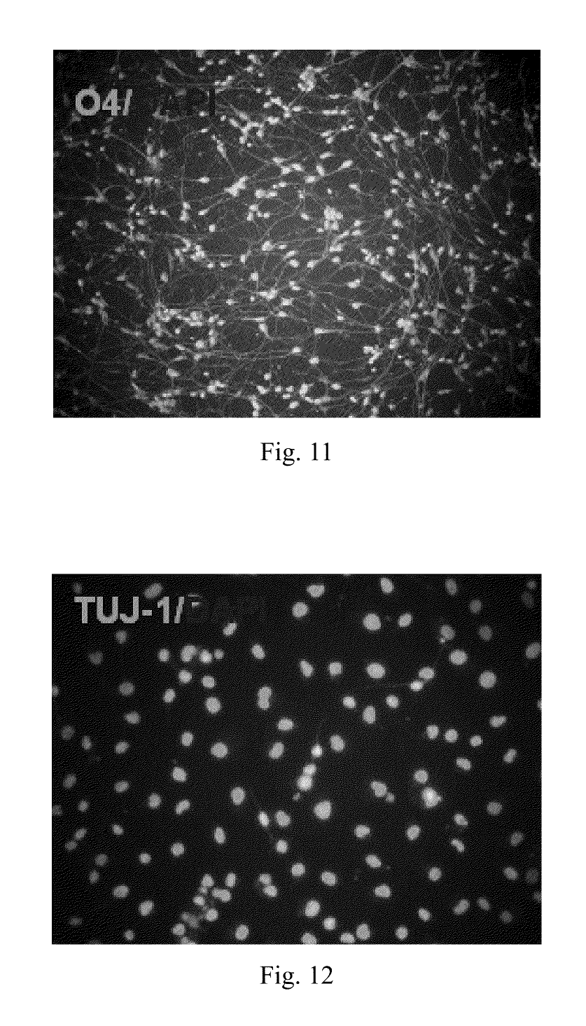

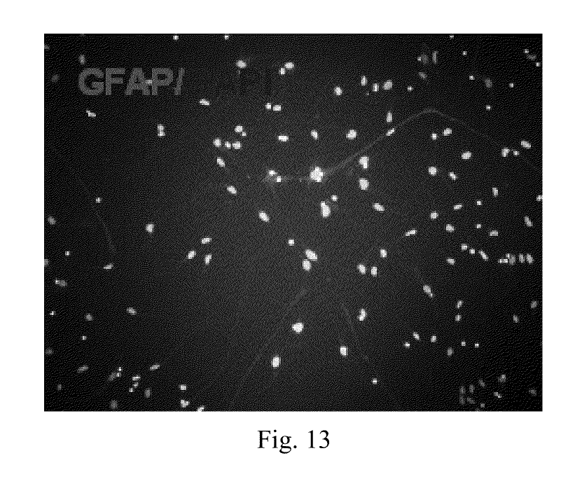

Then the OPCs of the tenth generation were processed with cell staining. As shown in FIGS. 11˜13 of the drawings, it can be seen that tuj-1 (neuronal marker) and GFAP (glial fibrillary acidic protein) are lowly expressed in the OPCs, and the marker O4 was highly expressed in the OPCs.

### EXAMPLE 2

Brain tissue was separated from brain of 10-week abortion embryos, and the brain tissue was dissociated into single cell mechanically, pre-treatment medium was added for culturing, and culture condition thereof was 37° C. with 8.5% CO₂ and saturated humidity. After the cells form a ball shape, passaged for OPCs induction.

Culture, induction, amplification and identification and method thereof were the same with the example 1. Identification result indicates that the OPCs induced thereby highly expressed OPCs markers such as O4 and NG2, and the OPCs markers had a positive rate of 80˜90%. The OPCs which were induced to the tenth generation remain an unchanged biological character.

### EXAMPLE 3

In this example, a second cell line of NSCs formed in our laboratory were utilized for inducing into OPCs. The NSCs were derived from cortex tissue of abandoned embryos. The NSCs were passaged to a 25th generation.

Culture, induction, amplification and identification and method thereof were the same with thereof the example 1. Identification result indicated that the OPCs induced thereby highly expressed OPCs markers such as O4 and NG2, and the OPCs markers had a positive rate of 80˜90%. The OPCs which were induced to the tenth generation remain an unchanged biological character.

Medium involved in the examples mentioned above and components thereof were as follows.

TABLE 1
Components of basal medium (DF medium)

| Components | Contents | |
|---|---|---|
| DMEM | 210 | ml |
| F12 | 70 | ml |
| HEPES | 15 | mM |

| | |
|---|---|
| D-glucose | 1.5% |

TABLE 2
Components of pre-treatment medium

| Components | Contents | |
|---|---|---|
| DF medium | 100 | ml |
| B27 | 2% | |
| bFGF | 10 | ng/ml |
| EGF | 20 | ng/ml |
| LIF | 10 | ng/ml |
| Transferin | 100 | μg/ml |
| Progerterone | 20 | nM |
| Putrescine | 60 | μM |
| Sodium Selenite | 30 | nM |
| Insulin | 25 | μg/ml |
| Heparin | 5 | μg/ml |

TABLE 3
Components of inducing medium for OPCs

| Components | Contents | |
|---|---|---|
| Neural Basal Medium or DF medium | 100 | ml |
| B27 | 2% | |
| Transferin | 5 | μg/ml |
| Progerterone | 10 | nM |
| Putrescine | 30 | μM |
| Sodium Selenite | 15 | nM |
| Insulin | 5 | μg/ml |
| Heparin | 2.5 | μg/ml |
| Sodium lactate | 5 | mM |
| bFGF | 5 | ng/ml |
| PDGF-AA | 10 | ng/ml |
| NT-3 | 10 | ng/ml |
| penicillin-streptomycin(optional)) | 100 | U/ml |

TABLE 4
Components of proliferating medium for OPCs

| Components | Contents | |
|---|---|---|
| Neural Basal Medium or DF medium | 50 | ml |
| Neural Basal Medium (sugar free) | 50 | ml |
| B27 | 2% | |
| Transferin | 5 | μg/ml |
| Progerterone | 10 | nM |
| Putrescine | 30 | μM |
| Sodium Selenite | 15 | nM |
| Insulin | 5 | μg/ml |
| Heparin | 2.5 | μg/ml |
| Sodium lactate | 5 | nM |
| bFGF ( | 10 | ng/ml |
| PDGF-AA | 10 | ng/ml |
| NT-3 | 10 | ng/ml |
| Penicillin-streptomycin(optional) | 100 | U/ml |

In the tables mentioned above, DMEM and F12 are common medium for cell culture, which are available in any commercial company. In the examples mentioned above, the DMEM and the F12 are purchased from Invitrogen Corporation,

wherein article number of the DMEM is 11965-118, and the specific formula thereof can be seen from the following website:

http://zh.invitrogen.com/site/cn/zh/home/support/Product-Technical Resources/media_formulation.8.html; and

wherein article number of the F12 is 11765-054, and the specific formula thereof can be seen from the following website:

http://zh.invitrogen.com/site/cn/zh/home/support/Product-Technical-Resources/media_formulation.64.html.

The Neural Basal Medium is common medium for neuronal cell culture, which is available in commercial companies. In the examples mentioned above, the Neural Basal Medium is purchased from Invitrogen Corporation, wherein article number thereof is 10888022, and the specific formula thereof can be seen from the following website:

http://www.lifetechnologies.com/cn/zh/home/technical-resources/media-formulation.253.html.

The sugar-free Neural Basal Medium is common medium for neuronal cell culture, which is available in commercial companies. In the examples mentioned above, the sugar-free Neural Basal Medium is purchased from Invitrogen Corporation, wherein article number thereof is 0050128DJ, and the specific formula thereof can be seen from the following website:

http://www.lifetechnologies.com/cn/zh/home/technical-resources/media-formulation.256.html.

The B27 is common additive for neuronal cell culture, which is available in commercial companies. In the examples mentioned above, the B27 is purchased from Invitrogen Corporation, wherein article number thereof is 17504-044, the specific formula can be seen from the following website:

http://zh.invitrogen.com/site/cn/zh/home/support/Product-Technical-Resources/media_formulation.250.html.

The EGF, bFGF, LIF, PDGF-AA and NT-3 are all common cytokines in cell culture, which are available in commercial companies. In the examples mentioned above, the EGF, bFGF, LIF, PDGF-AA and NT-3 are purchased from Peprotech Corporation, and article numbers thereof are respectively EGF: AF-100-15, bFGF: AF-100-18B, LIF: AF-300-05, PDGF-AA: 100-13A-10, NT-3: 450-03-10.

The sodium lactate, D-glucose, transferin, progerterone, putrescine, sodium selenite, sodium selenate, Insulin, heparin, trypsin and trypsin inhibitor were purchased from Sigma Corporation, and article numbers thereof were respectively: sodium lactate: L7022, D-glucose: G8644, transferin: T2036, progerterone: P8783, putrescine: P5780, sodium selenite: 55261, sodium selenate: 55261, insulin: 13536, heparin: H3149, trypsin: T4674 and trypsin inhibitor: T6522.

The HEPES was purchased from Corning Corporation, and article number thereof was 25-060-CI.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A differentiation and amplification method for inducing human neural stem/progenitor cells to differentiate into oligodendrocyte progenitor cells, comprising following steps of:
pre-treating neural stem/progenitor cells (NS/PCs) of human in pre-treatment medium for culturing a preset time,
inducing the NS/PCs after pre-treating with inducing medium, so as to differentiate into high-purity oligodendrocyte progenitor cells (OPCs) which are capable of expressing OPCs makers including O4, A2B5 and NG2,
wherein the inducing medium substantially comprises bFGF, PDGF-AA and NT-3,
wherein the OPCs obtained thereby are capable of proliferating steadily in proliferating medium for at least 10 generations,
wherein the proliferating medium substantially comprises bFGF, PDGF-AA, NT3 and sodium lactate.

2. The differentiation and amplification method for inducing human neural stem/progenitor cells to differentiate into oligodendrocyte progenitor cells, as recited in claim 1, specifically comprising following steps of:
1. dissociating NS/PCs of human into suspension of single cells;
2. suspending the single cells in a pre-treatment medium again after washing, wherein cell density thereof is adjusted to 2˜10×10⁵/ml;
3. planting cells suspension into a cell culture flask, and culturing under a condition of 37° C. with 5˜8.5% CO₂ and saturated humidity;
4. renewing a half of the medium every 3˜5 days until the cells are cultured continuously for 7˜12 days;
5. collecting the cells into a centrifuge tube, wherein 400 g thereof is processed with centrifugation for 5 minutes for precipitating the cells;
6. removing supernatant, wherein a mass percentage of 0.025% trypsin is applied for digesting the cells, in such a manner that the cells are digested into single cells suspension, after digesting for 10 minutes, a concentration of 1 mg/ml trypsin inhibitor is applied for inhibiting digestion;
7. centrifugating 400 g of suspension for 5 minutes to collect cells;
8. removing supernatant, wherein inducing medium for OPCs is applied to suspend the cells again, and cell density thereof is adjusted to 2˜10×10⁵/ml;
9. planting suspension of cells into a cell culture flask;
10. processing morphological identification on the OPCs and immunofluorescence staining identification on OPCs markers after inducing for 4˜10 days to find that a large quantity of cells are adhered on a bottom of the cell culture flask;
11. collecting the OPCs into a centrifuge tube;
12. centrifugating 400g of collection for 5 minutes to collect cells;
13. removing supernatant, wherein the cells are suspended in proliferating medium again, and cell density thereof is adjusted to 2˜10×10⁵/ml;
14. planting cell suspension into a cell culture flask;
15. renewing the proliferating medium for every 3˜5 days; and
16. after approximately one week when cell confluence of the OPCs reach 80%, processing passage on the OPCs, wherein passage method thereof is the same with the steps 11˜15 mentioned above, wherein the steps are cycled.

3. The differentiation and amplification method for inducing human neural stem/progenitor cells to differentiate into oligodendrocyte progenitor cells, as recited in claim 2, wherein the pre-treatment medium comprises basal medium and additives, wherein the basal medium is commercial Neural Basal Medium or self-prepared DF medium;
wherein the DF medium comprises: DMEM, F12, HEPES and D-glucose, a volume ratio of the DMEM and F12 is (1˜3): 1, a concentration of the HEPES is 10˜20 mmol/L, a concentration of the D-glucose is 1˜2 g/ml (mass to volume),
wherein components of the additives comprise B27, bFGF, EGF, LIF, transferin, progerterone, putrescine, sodium selenite, insulin and heparin, wherein mass concentrations are respectively: 1× of B27, 15-25 ng/ml of EGF, 10-20 ng/ml of bFGF, 7-13 ng/ml of LIF, 50-150 μg/ml of transferin, 10-30 mmol/L of progerterone, 50-150 μmol/L of putrescine, 20-40 mmol/L of sodium selenite, 10-50 μg/ml of insulin and 3-10 μg/ml of heparin.

4. The differentiation and amplification method for inducing human neural stem/progenitor cells to differentiate into oligodendrocyte progenitor cells, as recited in claim 2, wherein the inducing medium comprises basal medium and additives, wherein the basal medium is commercial Neural Basal Medium or self-prepared DF medium;
wherein the DF medium comprises DMEM, F12, HEPES and D-glucose, a volume ratio of the DMEM and F12 is (1˜3): 1, a concentration of the HEPES is 10˜20 mmol/L, a concentration of the D-glucose is 1˜2 g/ml (mass to volume),
wherein the additives comprise: B27, transferin, progerterone, putrescine, sodium selenite, insulin, heparin, sodium lactate, bFGF, PDGF-AA, and NT-3, and optionally penicillin-streptomycin, wherein mass concentrations are respectively: 1× of B27, 5-20 μg/ml of transferin, 5-20 nmol/L of progerterone, 20-40 μmol/L putrescine, 10-20 nmol/L of sodium selenite, 5-20 μg/ml of insulin, 2-10 μg/ml of heparin, 3-10mmol/L of sodium lactate, 5-30 ng/ml of bFGF, 5-30 ng/ml of PDGF-AA, 5-30 ng/ml of NT-3 and optionally 100 U/ml of penicillin-streptomycin.

5. The differentiation and amplification method for inducing human neural stem/progenitor cells to differentiate into oligodendrocyte progenitor cells, as recited in claim 2, wherein the proliferating medium comprises basal medium and additives, wherein the basal medium comprises commercial Neural Basal Medium or self-prepared DF medium, and commercial sugar-free Neural Basal Medium, wherein volume ratio of the commercial Neural Basal Medium or the self-prepared DF medium, and the commercial sugar-free Neural Basal Medium is (1˜3): 1;
wherein the DF medium comprises DMEM, F12, HEPES and D-glucose, a volume ratio of the DMEM and F12 is (1˜3): 1, a concentration of the HEPES is 10˜20 mmol/L, a concentration of the D-glucose is 1˜2 g/ml (mass to volume),
wherein the additives comprise B27, sodium lactate, bFGF, PDGF-AA, NT-3, transferin, progerterone, putrescine, sodium selenite, insulin and heparin, wherein mass concentrations are respectively: 1× of B27, 3-10 mmol/L of sodium lactate, 5-25 ng/ml of bFGF, 10-20 ng/ml of PDGF-AA, 5-25 ng/ml of NT-3, 5-50 μg/ml of transferin, 5-20 mmol/L of progerterone, 20-50 μmol/L of putrescine, 10-20 mmol/L of sodium selenite, 5-20 μg/ml of insulin and 2-10 μg/ml of heparin

6. The differentiation and amplification method for inducing human neural stem/progenitor cells to differentiate into oligodendrocyte progenitor cells, as recited in claim 1, wherein the human neural stem/progenitor cells are derived from brain tissue of human, spinal cord tissue, embryonic stem cells or induced pluripotent stem cells (iPS).

7. A method of preparing medicine for treating diseases of nervous system damage, comprising adding a therapeutically effective amount of the OPCs obtained according to the method of claim 1 thereinto.

8. A method of preparing medicine for treating diseases of nervous system damage, comprising adding a therapeutically effective amount of the OPCs obtained according to the method of claim 2 thereinto.
